(19) United States
(12) Patent Application Publication
(10) Pub. No.: US 2014/0316275 A1
(43) Pub. Date: Oct. 23, 2014

(54) HIGH FREQUENCY ULTRASONIC CONVEX ARRAY TRANSDUCERS AND TISSUE IMAGING

(71) Applicants: Hyung Ham Kim, Los Angeles, CA (US); Jin Ho Chang, Bucheon (KR); K. Kirk Shung, Monterey Park, CA (US)

(72) Inventors: Hyung Ham Kim, Los Angeles, CA (US); Jin Ho Chang, Bucheon (KR); K. Kirk Shung, Monterey Park, CA (US)

(73) Assignee: UNIVERSITY OF SOUTHERN CALIFORNIA, Los Angeles, CA (US)

(21) Appl. No.: 14/323,524

(22) Filed: Jul. 3, 2014

Related U.S. Application Data
(62) Division of application No. 12/680,504, filed on Mar. 26, 2010, now Pat. No. 8,310,316.
(60) Provisional application No. 60/975,616, filed on Sep. 27, 2007.

(51) Int. Cl. A61B 8/08; A61B 8/10; A61B 8/06

(52) U.S. Cl. CPC A61B 8/488; A61B 8/06; A61B 8/10
USPC 600/454

(57) ABSTRACT

A high frequency ultrasonic transducer may include a plurality of adjacent ultrasonic transducer elements. The adjacent transducer elements may be sized and configured so as to resonate at a frequency that is at least 15 MHz. The adjacent transducer elements may collectively form an aperture that is substantially convex along a lateral dimension spanning the cascaded width of the adjacent transducer elements. The aperture may be substantially concave along an elevation spanning the height of each of the transducer elements. The ultrasonic transducer and an associated transmitter system may be configured so as to enable ultrasound that is radiated from the plurality of the transducer elements to be focused on and to scan across locations that are no more than 30 millimeters from the aperture and that span across a field of view of at least 50 degrees without movement of the ultrasonic transducer or tissue during the scanning.

### CROSS-REFERENCE TO RELATED APPLICATIONS

This application is a divisional of U.S. patent application Ser. No. 12/680,504, filed Mar. 26, 2010, entitled “HIGH FREQUENCY ULTRASONIC CONVEX ARRAY TRANSDUCERS AND TISSUE IMAGING,” attorney docket no. 028080-0564; which is a national filing of PCT Application No. PCT/US2008/077857, filed 26 Sep. 2008, entitled “HIGH FREQUENCY ULTRASONIC CONVEX ARRAY TRANSDUCERS AND TISSUE IMAGING,” attorney docket no. 028080-0564; which claims the benefit of U.S. Provisional Application No. 60/975,616, entitled “SPECIALLY DESIGNED ARRAY TRANSDUCERS FOR HIGH FREQUENCY ULTRASOUND IMAGING,” filed Sep. 27, 2007, attorney docket number 28080-291.

The entire content of each of these applications is incorporated herein by reference in its entirety.

### STATEMENT REGARDING FEDERALLY SPONSORED RESEARCH

This invention was made with government support under Contract No. P41EB2181 awarded by the National Institutes of Health. The government has certain rights in the invention.

### BACKGROUND

1. Technical Field

This disclosure relates to ultrasonic transducers and imaging systems and, more particularly, to high frequency ultrasonic transducers and tissue imaging systems.

2. Description of Related Art

High frequency tissue imaging systems may be used to image ophthalmic tissue. These may be based on fixed-focus, single element transducers. Arc scanning may be used to image along the contour of the anterior segment of an eye. Sector scanning may be used to image the posterior segment of an eye. However, the aperture may have to be translated mechanically. This may result in a low frame rate.

Annular array transducers may achieve better spatial resolution over a larger depth of field. However, they may still need to be translated mechanically.

Doppler may be implemented by single element transducers or annular arrays. However, color flow mapping may be difficult to implement with these transducers.

Linear array transducers may achieve a higher frame rate with electronic translation. However, their field of view may be too narrow to image a wide area of tissue, such as a human eye, in one imaging plane.

### SUMMARY

A high frequency ultrasonic transducer may include a plurality of adjacent ultrasonic transducer elements. The adjacent transducer elements may be sized and configured so as to resonate at a frequency that is at least 15 MHz, 20 MHz, or 30 MHz. The adjacent transducer elements may collectively form an aperture that is substantially convex along a lateral dimension spanning the cascaded width of the adjacent transducer elements. The aperture may be substantially concave along an elevation spanning the height of each of the transducer elements.

The aperture may have a radius of curvature along the lateral dimension that is between 8 and 60 millimeters.

The aperture may have a radius of curvature along the elevation that is between 3 and 60 millimeters.

The height of each of the transducer elements may be between 2 and 12 millimeters.

The number of transducer elements may be between 60 and 300.

The transducer elements may be made from a piezoelectric material, such as 1-3 composite, high dielectric constant piezo ceramic.

An ultrasonic tissue imaging system may include an ultrasonic transducer comprising a plurality of ultrasonic transducer elements configured to collectively form an aperture, a transmitter system configured to generate and deliver a plurality of signals simultaneously to a plurality of the transducer elements, a receiver system configured to receive signals simultaneously from a plurality of the transducer elements and to perform receive focusing on these signals, and an imaging system configured to generate an image of tissue based on the receive focusing. The ultrasonic transducer and the transmitter system may be configured so as to enable ultrasound that is radiated from the plurality of the transducer elements to be focused on and to scan across locations that are no more than 75, 50 or 30 millimeters from the aperture and that span across a field of view of at least 20, 35 or 50 degrees without movement of the ultrasonic transducer or tissue during the scanning.

The imaging system may be configured to use the Doppler effect to generate information useful in evaluating blood flow in a vascular system. The imaging system may include a color flow system configured to generate color images that are indicative of the blood flow and/or a Doppler system configured to generate Doppler data that is indicative of the instantaneous or average velocity of the blood flow at a certain point.

A tissue imaging method may include positioning tissue to be imaged within no more than 75, 50 or 30 millimeters of an aperture of an ultrasonic transducer. While at this position and without moving the ultrasonic transducer or the tissue, the method may include causing the ultrasonic transducer to generate ultrasound that is focused on and that scans across a field of view of the tissue to be imaged of at least 20, 35 or 50 degrees. The method may include producing an image of the field of view of the tissue to be imaged of at least 20, 35 or 50 degrees based on reflections of the ultrasound from the tissue to be imaged that are received by the ultrasonic transducer.

The tissue to be imaged may be part of a human eye.

The tissue to be imaged may be a posterior segment of the human eye. The tissue imaging method may include diagnosing whether there is retina vein occlusion, macular degeneration, or retinal detachment in the human eye based at least in part on the image.

The tissue to be imaged may be an anterior segment of the human eye. The tissue imaging method may include diagnosing whether there is a cataract or hyphema in the human eye based at least in part on the image.

The tissue to be imaged may be a mouse heart.

The tissue imaging method may include guiding micro surgery based on the image.

The tissue imaging method may include evaluating the results of surgery based on the image.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings disclose illustrative embodiments. They do not set forth all embodiments. Other embodiments may be used in addition or instead. Details that may be apparent or unnecessary may be omitted to save space or for more effective illustration. Conversely, some embodiments may be practiced without all of the details that are disclosed. When the same numeral appears in different drawings, it is intended to refer to the same or like components or steps.

FIG. 1 illustrates a high frequency convex ultrasonic transducer array positioned to image a portion of a human eye.

FIG. 2 illustrates an aperture of a high frequency ultrasonic transducer array.

FIG. 3 is a block diagram of the front end of an ultrasonic imaging system.

FIG. 4 is a block diagram of a backend of an ultrasonic imaging system.

FIG. 5(a) illustrates simulated wire phantom images by a linear array.

FIG. 5(b) illustrates simulated wire phantom images by a convex array.

FIG. 6 is a grey scale H&E stain image of a dog's eye.

FIG. 7(a) is a simulated ultrasound image for the dog's eye illustrated in FIG. 6 using a linear array.

FIG. 7(b) is a simulated ultrasound image for the dog's eye illustrated in FIG. 6 using a convex array.

### DETAILED DESCRIPTION OF ILLUSTRATIVE EMBODIMENTS

Illustrative embodiments are now discussed. Other embodiments may be used in addition or instead. Details that may be apparent or unnecessary may be omitted to save space or for a more effective presentation. Conversely, some embodiments may be practiced without all of the details that are disclosed.

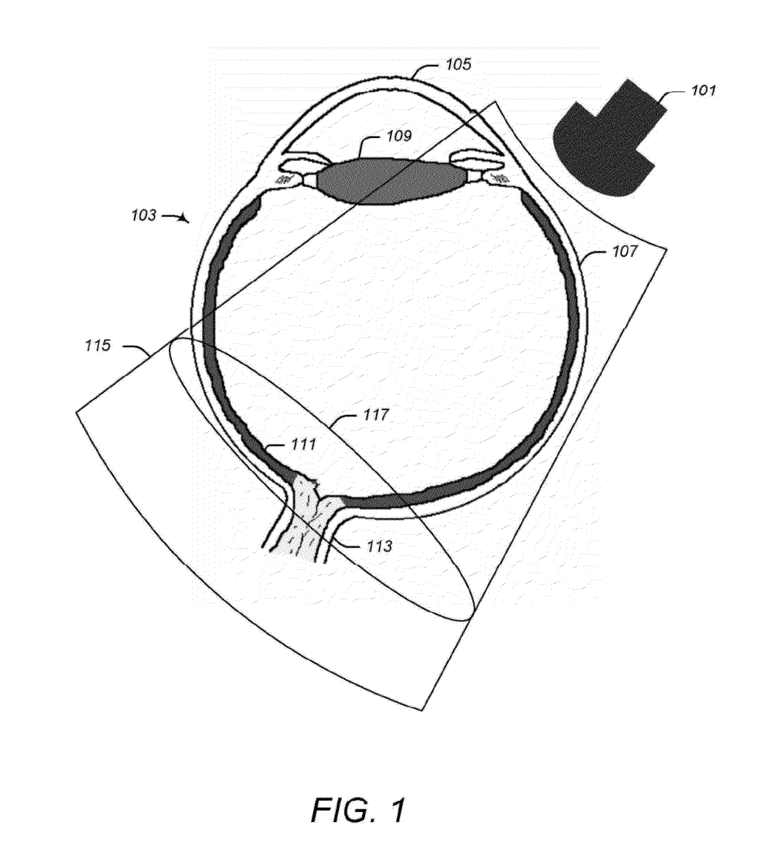

FIG. 1 illustrates a high frequency convex ultrasonic transducer array positioned to image a portion of a human eye.

As illustrated in FIG. 1, a high frequency convex ultrasonic transducer array 101 may be positioned in close proximity to a human eye 103, such as next to a portion of the sclera 107 of the human eye 103. The human eye 103 may be approximately one inch in diameter and may include a cornea 105, a lens 109, a retina 111, and an optic nerve 113.

The ultrasonic transducer array 101 may include a plurality of adjacent ultrasonic transducer elements. Any number of elements may be used. For example, there may be between 60 and 300 adjacent elements. In one embodiment, 192 adjacent elements may be used.

The elements may have any pitch. For example, the elements may have a pitch that varies between 0.5 and 1.5 times the wavelength of the signal that is used to drive the elements. In one embodiment, a pitch of 1.5 wavelength may be used.

The elements may be made of any material that generates ultrasound upon excitation. For example, a piezoelectric material may be used as the active material, such as a 1-3 composite with a high dielectric constant piezo ceramic. Two matching layers and a lossy low impedance backing layer may also be provided.

As illustrated in FIG. 1, the ultrasonic transducer array 101 may be close to but not touching the outer surface of the human eye 103. In this situation, a gel or package of gel may be placed between the surface of the ultrasonic transducer array 101 and the surface of the human eye 103, thus substantially filling the ultrasound pathway between the two.

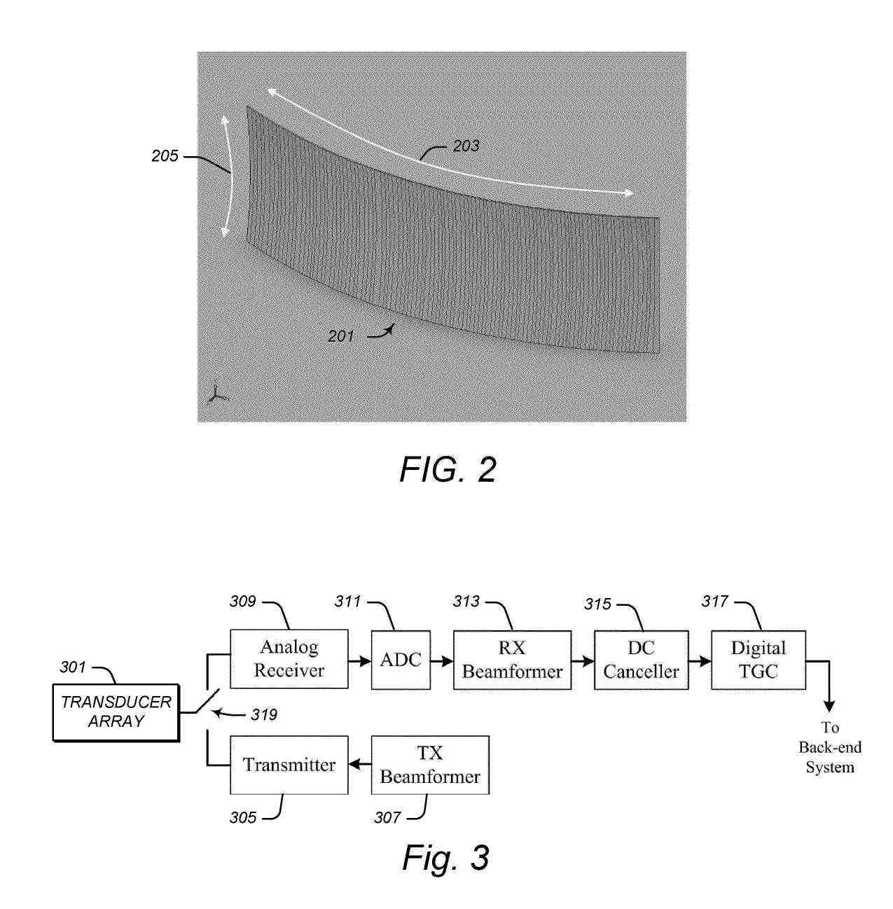

FIG. 2 illustrates an aperture 201 of a high frequency ultrasonic transducer array. This may be the aperture of the ultrasonic transducer array 101 illustrated in FIG. 1 or any other transducer. Conversely, the ultrasonic transducer array 101 that is illustrated in FIG. 1 may have an aperture which is different from the one illustrated in FIG. 2.

As illustrated in FIG. 2, the aperture 201 may be substantially convex along a lateral dimension 203 that spans the cascaded width of the adjacent transducer elements. These elements are illustrated in FIG. 2 as the narrow, vertical segments that make up the aperture. The aperture may also be concave along an elevation 205 that spans the height of each of the transducer elements.

The radius of the convex curvature along the lateral dimension 203 may vary depending upon the application. For example, the radius of convex curvature may be between 15 and 35 millimeters. In one embodiment, the radius of convex curvature may be approximately 24 millimeters.

The concave curvature along the elevation 205 may vary depending upon the application. For example, the concave curvature along the elevation 205 may have a radius of curvature between 3 and 60 millimeters . In one embodiment, the radius of curvature may be approximately 30 millimeters.

The height of each transducer element may vary depending upon the application. For example, the transducer elements may have a height that is between two and twelve millimeters. In one embodiment, the height of the transducer elements may be approximately seven millimeters.

The resonant frequency of the transducer elements and the frequency at which they are driven may vary depending upon the application. For example, the resonant and driving frequency may be at least 15 megahertz, at least 20 megahertz, or at least 30 megahertz. In one embodiment, a resonant and driving frequency of approximately 20 megahertz may be used.

The resonant and driving frequency which is ultimately selected, as well as the size, number, and curvatures of the ultrasonic transducer and elements may be selected based on a broad variety of considerations. These may include the desired spatial sampling, resolution, and permissible amount of aliasing. Field II software may be used to simulate the sound fields that may result to aid in their selection.

The ultrasonic transducer array 101 with the aperture 201 may be configured with a selected driving frequency to focus the ultrasound which the array generates on and to scan across locations that are located within an imaging plane 115 and a region of interest 117, as illustrated in FIG. 1.

The distances from the aperture of the ultrasonic transducer array 101 at which sound may be focused may vary. By appropriate selection of the driving frequency and structure of the ultrasonic transducer array 101, including the concave and convex curvatures of the aperture 201 discussed above, the ultrasound that is generated by the ultrasonic transducer array 101 may be able to focus on tissue that is located within 75 millimeters or less from the aperture of the transducer. The sound may also be able to focus on tissue that is within 30 millimeters or less of the aperture.

The field of view across which the ultrasound generated by the ultrasonic transducer array 101 may focus thorough signal driving manipulation without movement of the ultrasonic transducer or the tissue to be imaged may also vary. With appropriate selections, the field of view may be at least 20 degrees, at least 35 degrees, or at least 50 degrees. In one embodiment, a field of view of 52 degrees may be realized.

As illustrated in FIG. 1, these short distances from the aperture at which the ultrasound may be focused, and these wide fields of view through which ultrasound may be scanned while in focus and without moving the transducer or tissue, may enable the ultrasonic transducer array 101 to accurately scan different portions of the human eye, such as an anterior segment of the human eye and/or a posterior segment of the human eye.

FIG. 3 is a block diagram of the front end of an ultrasonic imaging system. The front end may be configured to transmit ultrasound, receive echo signals, and improve signal-to-noise ratio.

As illustrated in FIG. 3, a transducer array 301 may be alternately connected between a transmitter system that may include a transmitter 305 and a transmit (TX) beamformer 307, and a receiver system that may include an analog receiver 309, an analog-to-digital converter (ADC) 311, a receive (RX) beamformer 313, a DC canceller 315, and a digital time-gain compensation (TGC) system 317. The transducer array 301 may be rapidly switched between the transmitter system and the receiver system using an electronic switch 319 operating under appropriate control circuitry.

On transmit, the transducer array 301 may be electronically focused, typically at a fixed imaging depth. In order to do so, the transmit (TX) beamformer 307 may be configured to calculate the needed time delay for each active element of the array transducer, and the transmitter 305 may be configured to excite each element following this predetermined time delay. The transducer array may be the ultrasonic transducer array 101 and may have the aperture 201 discussed above, or may be different transducer and/or may have a different aperture.

The analog receiver 309 may be configured to amplify signals that are received by the transducer array 301. The analog receiver 309 may include preamplifiers that are positioned close to the transducer array 301. The gain may be determined based on the sensitivity of the transducer array 301 and the input level required by the analog-to-digital converter (ADC) 311.

Digitized echo signals from each element may be sent to the receive (RX) beamformer 313. The receive (RX) beamformer 313 may be configured to perform receive focusing.

Focused echo signals from the receive (RX) beamformer may contain a DC component. However, envelope detection may need to be carried out based on echo signals that do not have a DC component. The DC canceller 315 may be configured to remove this DC component.

The digital time-game compensation (TGC) system 317 may be configured to cooperate with analog time-gain compensation in the analog receiver 309 to increase the amplitude of the echo signals along with imaging depth. This may compensate for energy loss caused by ultrasound attenuation and beam diffraction.

The transmitter 305 may be configured to simultaneously drive all or only some of the transducer elements in the transducer array 301. For example, the transmitter 305 may be configured to simultaneously drive approximately ⅓ of the elements in the transducer array. When the transducer array 301 contains 192 adjacent transducer elements, for example, the transmitter 305 may be configured to drive 64 of these adjacent elements simultaneously, followed by the next set of 64 adjacent elements, followed by the last set of 64 adjacent elements. The analog receiver 309 may similarly be configured to process echoes that are received by the corresponding transducer elements simultaneously, and to correspondingly shift to the remaining sets of transducer elements sequentially, in lock-step with the transmitter 305.

The transmitter system and the receiver system may be configured much differently than is illustrated in FIG. 3. For example, transmitted ultrasound may be in short or elongated pulses. Especially, elongated pulses may include phase codes like Barker or Golay codes with or without modulation by using a carrier signal and chirp code generated by linear frequency modulation (FM). The use of these elongated pulses may allow increasing penetration depth. For this coded excitation technique, a compression block with either matched or mismatched filters, called a decoder, may be placed in right after the ADC or the RX beamformer.

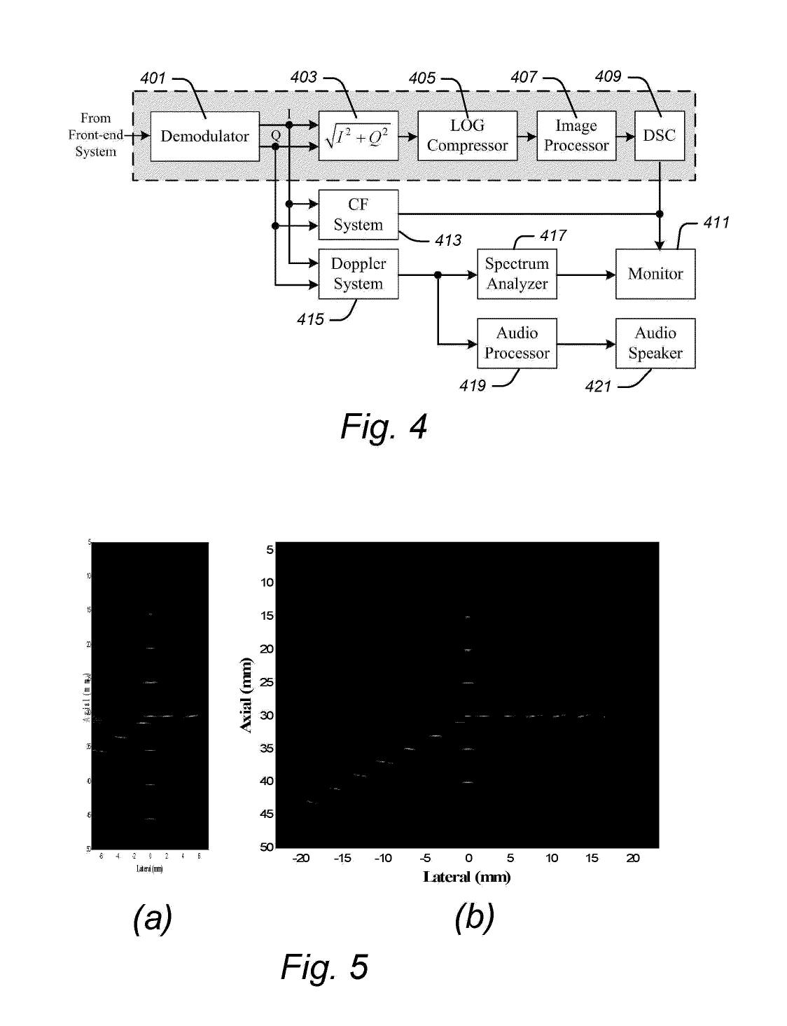

FIG. 4 is a block diagram of a back end of an ultrasonic imaging system. This back end may be used with the front end illustrated in FIG. 3 or with a different front end. Similarly, the front end illustrated in FIG. 3 may be used with a back end that is different than the one illustrated in FIG. 4.

The back end may be configured to extract clinically useful information from the echo signals that are generated from the front end system. For B-Mode imaging, for example, an envelope detector, including a demodulator 401 and a square root computational system 403 may be used, along with a logarithmic (LOG) compressor 405, an image processor 407, and a digital scan converter (DSC) 409. This processed information may be delivered to a monitor 411.

Clinically meaningful information about tissue may be contained in the envelope variation of echo signals arising from different tissues. The envelope detector may perform the function of removing the carrier signal and computing envelope values from echo signals. The enveloped echo signals may be logarithmically compressed in the logarithmic (LOG) compressor 405 for efficient visualization.

The transducer array 301 and the analog receiver 309 may respond to a wide range in the amplitude of echo signals, such as over 100 dB. The systems before the logarithmic (LOG) compressor 405 may be configured to also have this large dynamic range in order to receive very weak signals attenuated from objects positioned at a deep depth in an imaging plane.

On the other hand, the dynamic range of a monitor 411 may be only about 40 dB. Yet, the clinically meaningful amplitude variations of echo signals may be at least 60 dB. So these may not be directly displayed on the monitor 411 without information loss.

The logarithmic (LOG) compressor 405 may address this problem. Small amplitude signals may be raised by reducing the large dynamic range in the logarithmic (LOG) compressor 405. This may attenuate them for the monitor 411, yet still allow the retention of clinically useful information.

After logarithmic compression by the logarithmic (LOG) compressor 405, the image processor 407 may carry out focal zone blending, edge enhancement, auto gain control (AGC), black hole/noise spike filtering, lateral filtering, and/or persistence in order to achieve high image quality. These may be employed in high-end ultrasound imaging systems to generate images with superior contrast, spatial resolution, and image uniformity.

The manipulated echo signals may be mapped onto pixels of the monitor 411 following echo amplitude v. gray scale conversion. However, each sample point of the echo signal may not be directly mapped onto each pixel in the monitor 411 because its spatial location may not correspond to a pixel in the monitor 411. This mismatching may be especially serious when sector scanning is used, since samples may be acquired in a polar coordinate system, while the pixels in the monitor 411 may be organized in a Cartesian coordinate system. Under this circumstance, scanned conversion processing may be used to find appropriate pixel values from the echo samples through coordinate transformation and data interpolation.

A color flow (CF) system 413 and a Doppler system 415 may be configured to use the Doppler effect to evaluate a vascular system in a non-invasive way. Two-dimension color flow images may be generated and may provide both the direction and the mean velocity of blood flow by different colors and their intensity, respectively. The information may be represented in different ways. For example, red and blue colors may be used to indicate blood flow toward and away from the transducer array 301, respectively. The shade of a color may be used to indicate the magnitude of the blood flow speed. The color flow (CF) system 413 may be combined with a B-mode imaging system that is capable of providing anatomical and blood flow information on clinical problems, such as jets from the stenotic vessels and leaking heart valves, flow reduction, and occlusion from atherosclerotic plaque.

Unlike the 2-D color flow (CF) system 413, the Doppler system 415 may be configured to obtain instantaneous or averaged blood flow velocity at a certain point, such as the range gate in pulsed wave (PW) mode or at an intersection point of transmit and receive beams in continuous wave (CW) mode. Doppler data may be transformed into frequency domain in a spectrum analyzer 417. The Doppler spectrum data may show the variation of flow velocity along time. An audio processor 419 may be used in conjunction with an audio speaker 421 to convert the Doppler data into sound. The color flow (CF) system 413 and/or the Doppler system 415 may be used with contrast agents to aid in connection with molecular imaging.

In one configuration, a 192 element convex ultrasonic array transducer may resonate and be driven at approximately 20 MHz. It may be positioned near a human eyeball having approximately a one-inch diameter. An approximately 1.5 wavelength pitch, 24 millimeter radius of curvature, and 52 degree maximum viewing angle may be chosen to provide adequate spatial sampling and resolution along with minimal image aliasing. Approximately a 7.0 millimeter elevation width and a 30 millimeter geometric focus may be chosen as a compromise between depth of field and resolution. The number of channels used to require one scan line may be 64.

Based on these selections, transmit and receive sound fields may be simulated by Field II software. For KLM modeling of a single array element, a 1-3 composite with a high dielectric constant piezo ceramic may be chosen as the active material, along with two matching layers and a lossy low impedance backing layer.

Based on these parameters and a single transmit focus and dynamic receive focusing, Field II simulation software predicts a −6 dB lateral and axial beam width of 200 μm and 50 μm, respectively. The −6 dB depth of focus is 4.8 mm. The grating lobe level is −75 dB at 20 degrees at a range of 30 mm. KLM Modeling shows an echo fractional bandwidth above 60 percent and sensitivity above −50 dB with reference to 1 VN.

FIG. 5(a) illustrates simulated wire phantom images by a linear array. FIG. 5(b) illustrates simulated wire phantom images by a convex array. As illustrated by a comparison of FIG. 5(a) with FIG. 5(b), the convex array produces a much wider field of view.

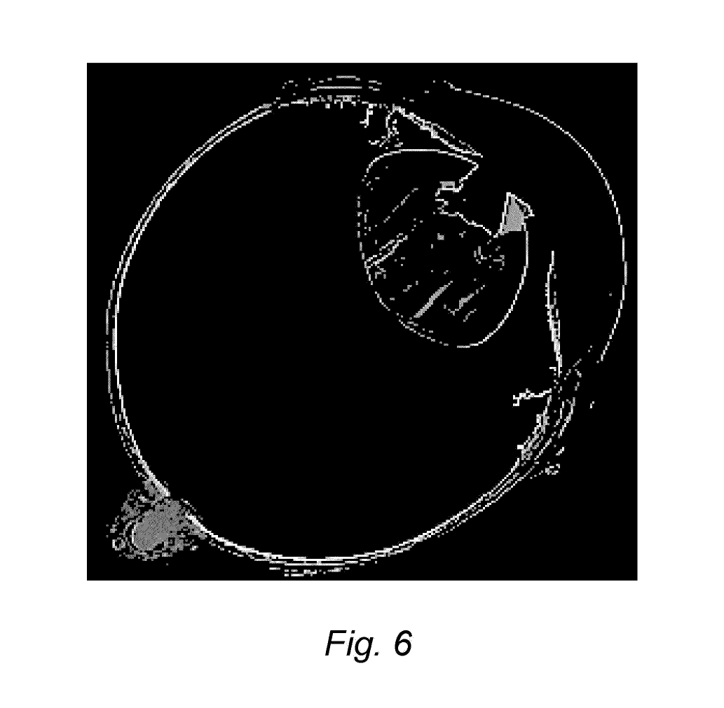

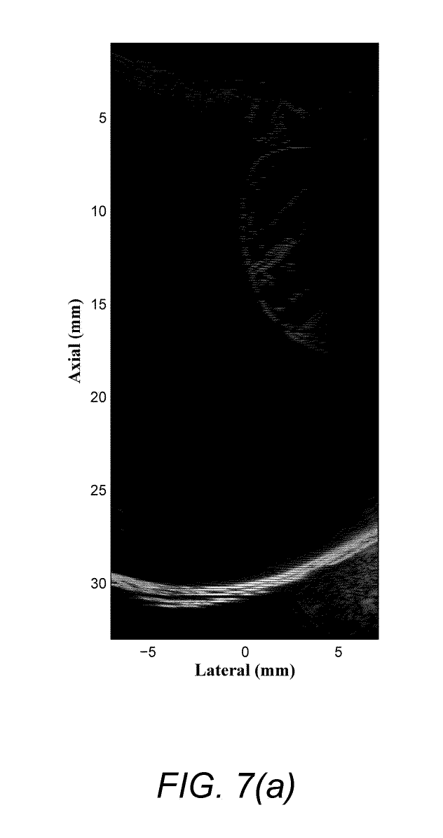

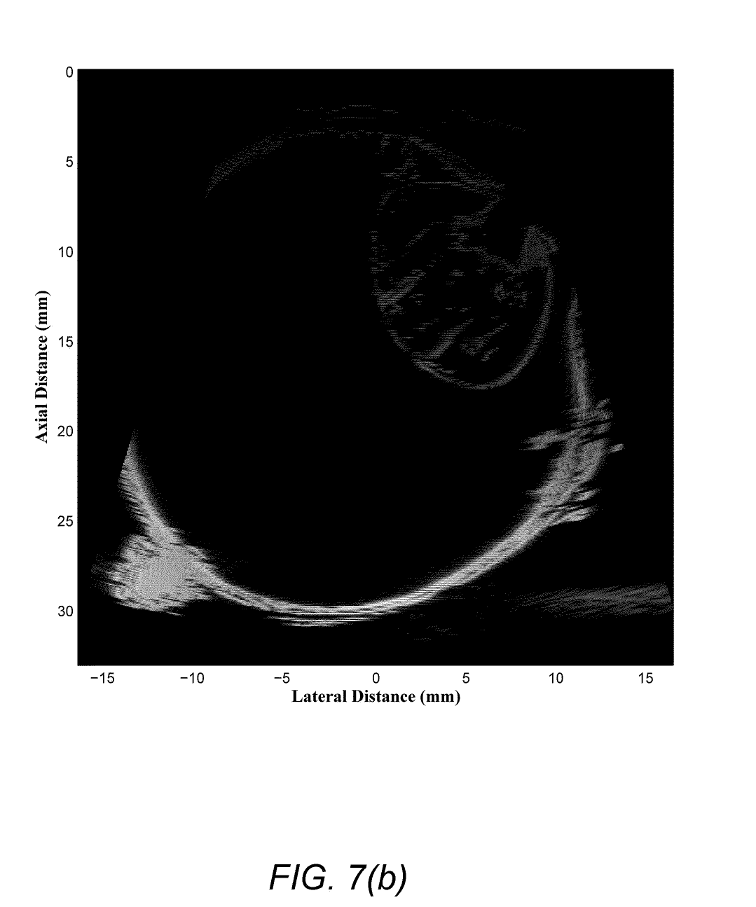

FIG. 6 is a grayscale H&E stain image of a dog's eye. FIG. 7(a) is a simulated ultrasound image for the dog's eye illustrated in FIG. 6 using a linear array. FIG. 7(b) is a simulated ultrasound image for the dog's eye illustrated in FIG. 6 using a convex array. Again, the ability of the convex array to produce a wider area of view without movement of the array or tissue is well illustrated by a comparison of these two figures.

The use of a high-frequency convex ultrasonic transducer array may cover an entire organ with a single image, achieve higher frame rates, create multiple focal zones with dynamic aperture, and/or implement Doppler and/or color flow mapping.

The design specification, including the center frequency, radius of curvature, number of elements, pitch, array length, array width, and other fabrication parameters may be changed based on the size, type and location of the tissue to be imaged. Higher frequencies may be used for applications that require higher spatial resolution with lower penetration.

High frequency ultrasonic convex transducer arrays may support a broad variety of applications that may not have been possible with low frequency ultrasound transducers and/or high frequency single element, annular array, and/or linear array transducers. For example, the posterior segment of a human eye may be imaged using this technology and used to help diagnose retinal vein occlusion, macular degeneration, and/or retinal detachment. Similarly, an anterior segment of a human eye may be imaged using this technology and used to help diagnose a cataract and/or hyphema.

These imaging systems may also be useful in other applications, such as to image a mouse heart in an adult mouse during experiments, to help guide micro-surgery using real-time imaging, and/or to help evaluate the results of surgery on-site after the surgery is complete.

The components, steps, features, objects, benefits and advantages that have been discussed are merely illustrative. None of them, nor the discussions relating to them, are intended to limit the scope of protection in any way. Numerous other embodiments are also contemplated, including embodiments that have fewer, additional, and/or different components, steps, features, objects, benefits and advantages. The components and steps may also be arranged and ordered differently.

The phrase “means for” when used in a claim embraces the corresponding structures and materials that have been described and their equivalents. Similarly, the phrase “step for” when used in a claim embraces the corresponding acts that have been described and their equivalents. The absence of these phrases means that the claim is not limited to any of the corresponding structures, materials, or acts or to their equivalents.

Nothing that has been stated or illustrated is intended to cause a dedication of any component, step, feature, object, benefit, advantage, or equivalent to the public, regardless of whether it is recited in the claims.

In short, the scope of protection is limited solely by the claims that now follow. That scope is intended to be as broad as is reasonably consistent with the language that is used in the claims and to encompass all structural and functional equivalents.

We claim:

1. An ultrasonic tissue imaging system comprising:
a high frequency ultrasonic transducer comprising a plurality of adjacent, linear ultrasonic transducer elements that resonate at a frequency that is at least 15 MHz and that collectively form an aperture that is substantially convex along a lateral dimension spanning the cascaded width of the adjacent, linear transducer elements;
a transmitter system configured to generate and deliver a plurality of signals simultaneously to a plurality of the transducer elements;
a receiver system configured to receive signals simultaneously from a plurality of the transducer elements and to perform receive focusing on these signals; and
an imaging system configured to generate an image of tissue based on the receive focusing,
wherein the ultrasonic transducer and the transmitter system enable ultrasound that is radiated from the plurality of the transducer elements to be focused on and to scan across locations that are no more than 75 millimeters from the aperture and that span across a field of view of at least 20 degrees without movement of the ultrasonic transducer or tissue during the scanning.

2. The ultrasonic tissue imaging system of claim 1 wherein the field of view is at least 35 degrees.

3. The ultrasonic tissue imaging system of claim 1 wherein the field of view is at least 50 degrees.

4. The ultrasonic tissue imaging system of claim 1 wherein the locations are no more than 50 millimeters from the aperture.

5. The ultrasonic tissue imaging system of claim 1 wherein the locations are no more than 30 millimeters from the aperture.

6. The ultrasonic tissue imaging system of claim 1 wherein the imaging system is configured to use the Doppler effect to generate information useful in evaluating blood flow in a vascular system.

7. The ultrasonic tissue imaging system of claim 6 wherein the imaging system includes a color flow system configured to generate color images that are indicative of the blood flow.

8. The ultrasonic tissue imaging system of claim 6 wherein the imaging system includes a Doppler system configured to generate Doppler data that is indicative of the instantaneous or average velocity of the blood flow at a certain point.

9. A tissue imaging method for imaging tissue using a high frequency ultrasonic transducer that includes a plurality of adjacent, linear ultrasonic transducer elements that resonate at a frequency that is at least 15 MHz and that collectively form an aperture that is substantially convex along a lateral dimension spanning the cascaded width of the adjacent, linear transducer elements, the method comprising:
positioning tissue to be imaged within no more than 75 millimeters of the aperture of the ultrasonic transducer;
while at this position and without moving the ultrasonic transducer or the tissue, causing the ultrasonic transducer to generate ultrasound that is focused on and that scans across a field of view of the tissue to be imaged of at least 20 degrees; and
producing an image of the field of view of the tissue to be imaged of at least 20 degrees based on reflections of the ultrasound from the tissue to be imaged that are received by the ultrasonic transducer.

10. The tissue imaging method of claim 9 wherein the field of view is at least 35 degrees.

11. The tissue imaging method of claim 9 wherein the field of view is at least 50 degrees.

12. The tissue imaging method of claim 9 wherein the tissue to be imaged is within no more than 50 millimeters of the aperture.

13. The tissue imaging method of claim 9 wherein the tissue to be imaged is within no more than 30 millimeters of the aperture.

14. The tissue imaging method of claim 9 wherein the tissue to be imaged is part of a human eye.

15. The tissue imaging method of claim 14 wherein the tissue to be imaged is a posterior segment of the human eye.

16. The tissue imaging method of claim 15 further comprising diagnosing whether there is retina vein occlusion in the human eye based at least in part on the image.

17. The tissue imaging method of claim 15 further comprising diagnosing whether there is macular degeneration in the human eye based at least in part on the image.

18. The tissue imaging method of claims 15 further comprising diagnosing whether there is retinal detachment in the human eye based at least in part on the image.

19. The tissue imaging method of claim 14 wherein the tissue to be imaged is an anterior segment of the human eye.

20. The tissue imaging method of claim 19 further comprising diagnosing whether there is a cataract in the human eye based at least in part on the image.

21. The tissue imaging method of claim 19 further comprising diagnosing whether there is hyphema in the human eye based at least in part on the image.

22. The tissue imaging method of claim 14 wherein the tissue to be imaged is a mouse heart.

23. The tissue imaging method of claim 14 further comprising guiding micro surgery based on the image.

24. The tissue imaging method of claim 14 further comprising evaluating the results of surgery based on the image.
